(19) United States
(12) Plant Patent Application Publication
(10) Pub. No.: US 2003/0172427 P1
(43) Pub. Date: Sep. 11, 2003

(54) Schefflera plant named ' Dazzle'

(76) Inventor: Sally L. Stribling, Homestead, FL (US)

Correspondence Address: FOLEY AND LARDNER, SUITE 500, 3000 K STREET NW, WASHINGTON, DC, 20007, US

(21) Appl. No.: 10/091,512

(22) Filed: Mar. 7, 2002

(51) Int. Cl. A01H 5/00

(52) U.S. Cl. PLT/377

(57) ABSTRACT

A new and distinct cultivar of Schefflera arboricola named ‘Dazzle’ as shown and described, and particularly characterized by the uniqueness of combined characteristics of heavily variegated foliage, large oblanceolate-shaped leaves with a cuspidate tip approximately 15-20 centimeters in length.

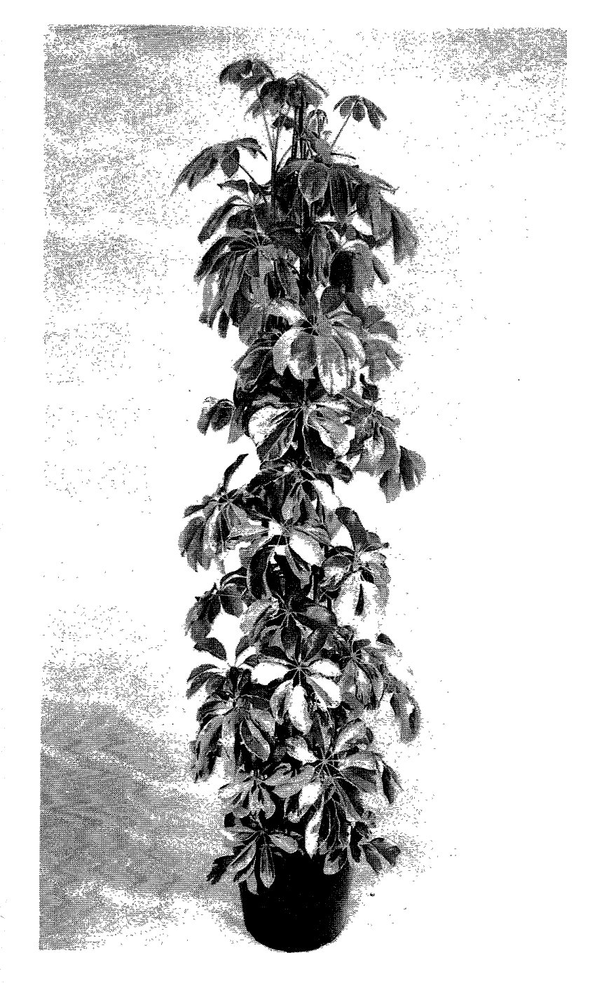

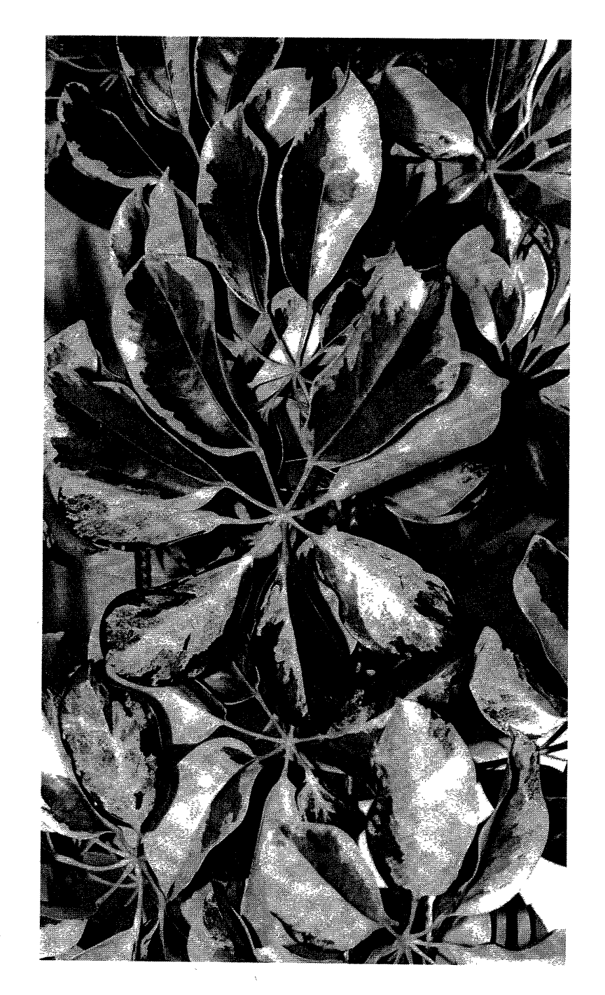

### BACKGROUND OF THE INVENTION

The present invention comprises a new and distinct cultivar of Schefflera plant, hereinafter referred to by the cultivar name ‘Dazzle’. ‘Dazzle’ is sport mutation from an unnamed cultivar of Schefflera arboricola.

The new cultivar was discovered by the inventor, Sally L. Stribling, in Homestead, Fla. as part of a controlled breeding program of Schefflera arboricola, as a sport mutation. The mutation was observed as a single branch growing from one plant of the unnamed parent cultivar (unpatented). The mutated branch was propagated by peat media stem cuttings in Homestead, Fla. under mist in a greenhouse.

The new characteristics of the new cultivar ‘Dazzle’ have remained constant through successive generations and the new cultivar reproduces true to type. Asexual reproduction through succeeding generations has established that the combination of characteristics as herein disclosed for the new cultivar are firmly fixed and retained through successive generations of asexual reproduction. The new invention differs substantially from the parent cultivar in leaf size, shape and leaf color.

### BRIEF SUMMARY OF THE INVENTION

The new cultivar differs from the parent plant in that the leaves are larger than that of Schefflerra arboricola and are somewhere in between the leaf size of the Schefflera Actinophylla and the parent plant, Schefflera arboricola.

The leaves of the new cultivar like those of the parent plant are smooth edged and are lacking serration. The new cultivar's leaf shape is oblanceolate where the parent plant is more obovate with an acuminate tip. Additionally the new cultivar has heavily variegated leaf coloration where the parent plant has no variegation.

No claims are made particularly to insect or disease resistance. The new cultivar is susceptible to aphids, scales and spider mites to a similar degree as is the parent plant.

To date the new cultivar ‘Dazzle’ has not born seed or fruit. S. arboricola (parent) generally bear seed after three years but the asexually propagated new cultivar dazzle has only been cultured to date for 18 months.

‘Dazzle’ has not been observed under all possible environmental conditions. The phenotypic expression may vary with variations in environment such as temperature, light intensity, day length and cultural conditions, without a change in the genotype of the plant.

### BRIEF DESCRIPTION OF THE DRAWING

The accompanying photographic drawing illustrates the new cultivar at one year and show as clearly as it is reasonably possible the plant form and foliage color that distinguish the new cultivar from the parent plant, Schefflera arboricola, and from other cultivars of this species. The drawing depicts a mature specimen of the new cultivar ‘Dazzle’ that has been grown trellised in a shadehouse in Homestead, Fla. under 72% shade during the months of January 2001 through December 2001.

### DETAILED BOTANICAL DESCRIPTION

In this description, color references are to The Royal Horticultural Society Colour Chart (RHS). The color references are approximate as color depends to a degree on horticultural practices such as light level and fertilization rates. The plant described is one year old.

Foliage

Leaf Shape.—Oblanceolate radially compound leaves with a cuspidate leaf tip.

Average Leaf Size.—15-20 cm in length and width, with approximately 8 leaflets per leaf measuring approximately 10 cm long and 5 cm wide.

Leaf texture.—Leathery, thick, smooth and glabrous.

Growth habit.—Upright, bushy, and full. Removal of terminal apices enhances branching.

Color of upper surface of foliage.—Heavily variegated coloration irregularly patterned, yellow-green RHS 147 A, green 139 A, yellow 11 B, gray 194 A.

Color of lower surface of foliage.—Mottled irregular variegation of green RHS 138 A and yellow-green RHS 144 C.

Stem color.—Older mature stem: RHS 144 A Juvenile stem: RHS 144 B.

Internode Length.—2.5-4 cm.

Leaflets per rack.—Approximately 8.

Plant height.—(as depicted in the first drawing): 180 cm at 1 year old from three rooted cuttings originally measuring 12 cm.

Plant Vigor.—Moderate, growth of 15 cm a month.

I claim:

1. A new and distinct cultivar of Schefflera arboricola named ‘Dazzle’ as herein shown and described.
